(19) United States
(12) Patent Application Publication
(10) Pub. No.: US 2026/0263745 A1
(43) Pub. Date: Sep. 10, 2026

(54) APPARATUS AND METHOD FOR TARGETED DELIVERY OF THROMBOLYTIC AGENT TO AN INDWELLING IV CATHETER TIP

(71) Applicant: Becton, Dickinson and Company, Franklin Lakes, NJ (US)

(72) Inventors: Manish Kumar, Bengaluru, Karnataka (IN); Ajay Suryavanshi, Mumbai, Maharashtra (IN); Praveen Nalawade, Belgaum, Karnataka (IN); Karthik MR, Bengaluru, Karnataka (IN); Shishir Prasad, Ramsey, NJ (US)

(73) Assignee: Becton, Dickinson and Company, Franklin Lakes, NJ (US)

(21) Appl. No.: 19/491,271

(22) PCT Filed: Jul. 30, 2024
(86) PCT No.: PCT/US2024/040103

§ 371 Date: Dec. 8, 2025

Related U.S. Application Data
(60) Provisional application No. 63/530,799, filed on Aug. 4, 2023.

(51) Int. Cl. A61M 25/01; A61M 25/00; A61M 39/10; A61M 39/24

(52) U.S. Cl. CPC A61M 25/0113; A61M 25/007; A61M 39/10; A61M 39/24; A61M 2025/0008; A61M 2039/1027; A61M 2039/242; A61M 2202/0007; A61M 2202/0014; A61M 2202/04; A61M 2205/0216; A61M 2205/3331; A61M 2205/583; A61M 2210/12

(57) ABSTRACT

Provided herein is an instrument delivery device for targeted delivery of a thrombolytic agent into an IV catheter. The instrument delivery device includes a collapsible sleeve and a catheter tube positioned within the collapsible sleeve. Proximal and distal connectors are positioned at the proximal and distal ends of the collapsible sleeve. An advancement member is positioned at the distal end of the collapsible sleeve, between the distal connector and the collapsible sleeve. The advancement member includes a housing, a wheel member on the housing and actuatable by a user, and a roller mechanism operably connected to the wheel member, with the catheter tube routed through the roller mechanism and engaged therewith. Rotation of the wheel member distally advances the catheter tube from a first position to a second position, in which the distal end of the catheter tube is disposed at or adjacent a distal end of the IV catheter.

### CROSS-REFERENCE TO RELATED APPLICATION

The present application claims priority to U.S. Provisional Application No. 63/530,799 entitled “Apparatus and Method for Targeted Delivery of Thrombolytic Agent to an Indwelling IV Catheter Tip” filed Aug. 4, 2023, the disclosure of which is hereby incorporated by reference in its entirety.

### BACKGROUND OF THE INVENTION

### Field of the Invention

The present disclosure relates generally to instrument delivery devices for use with intravenous (IV) catheters and, more specifically, to instrument delivery devices that provide for targeted delivery of a thrombolytic agent to the tip of an indwelling IV catheter.

### Description of Related Art

Vascular access devices (VADs) are used in the medical field to access peripheral and/or central vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

The use of IV catheters for purposes of infusion therapy and/or blood withdrawal is ubiquitous in the healthcare industry. For example, more than 27 million CVC insertion procedures are performed globally per year, with more than 5 million CVC insertion procedures performed in the United States alone. Additionally, over 2.7 million PICC insertions are performed yearly in the United States.

One of the most common complications associated with PICC and CVC insertions is the issue of catheter occlusion or blockage. Catheter occlusion affects 1 in every 8 PICCs (12.5%) placed and 1 in every 4 CVCs (25%) placed-with 58% of catheter occlusions being thrombotic occlusions that result from the formation of a thrombus clot within, surrounding, or at the tip of the catheter. As a result of such catheter occlusions, a PICC or CVC may need to be removed from the patient, with it being recognized that such catheter removal is time consuming, uncomfortable for the patient, and expensive due to increased length of stay and therapy interruptions.

Due to these aforementioned drawbacks associated with catheter removal, it is desirable to opt for treatment of the thrombotic occlusion rather than remove the catheter from the patient. Treatment of a thrombotic occlusion is typically achieved via the use of a thrombolytic agent, i.e., a tissue plasminogen activator (TPA), with alteplase, reteplase, and tenecteplase being non-limiting examples of TPAs typically used for removal of thrombotic occlusions. The TPA is typically instilled in the lumen of the IV catheter at a concentration of 2 mg/2 ml, for example, to treat the thrombotic occlusion, with it recognized that the concentration is limited due to the risk of bleeding if the concentration is too high. While TPA is effective for removal of the thrombotic occlusion, the targeted local delivery of the TPA to the tip of the IV catheter is challenging, especially with a fully-occluded catheter, as a clotted/occluded catheter “vacuum locks” the intraluminal fluid, i.e., the TPA, such that the TPA cannot flow all the way to the clot, but instead relies on diffusion (dilution) to reach the clot at the catheter tip. Accordingly, it may require several doses or treatments of TPA to fully remove the clot, as a success rate of single dose of TPA is typically around 50% and a success rate of second dose of TPA is typically around 80%. The need for multiple doses of TPA is both costly and time consuming, as each 2 mg dose may cost $125-150 and as each dose requires a 30 to 120 min wait time for the TPA to act on the clot.

Accordingly, a need exists in the art for an apparatus and method that provides for targeted delivery of a thrombolytic agent to the tip of an indwelling IV catheter. The apparatus and method would enable local delivery of the agent at the site of clot formation at/near the catheter tip, thereby providing a longer dwell-time of the agent at the target site before it diffuses/equilibrates along the entire catheter length, resulting in a faster thrombolytic interaction between the agent and the fibrin clot, so to reduce a wait time for removal of the occlusion and eliminate the need for application of a second dose of the agent.

### SUMMARY OF THE INVENTION

Provided herein is an instrument delivery device for targeted delivery of a thrombolytic agent into an intravenous (IV) catheter of a vascular access device. The instrument delivery device includes a collapsible sleeve comprising a distal end and a proximal end, and a catheter tube positioned at least partially within the collapsible sleeve and coaxially therewith, with the catheter tube defining a lumen therein and including a distal end and a proximal end, the distal end comprising a blunt tip having a slit formed therein. The instrument delivery device also includes a proximal connector positioned at the proximal end of the collapsible sleeve, with the proximal end of the collapsible sleeve and the proximal end of the catheter tube coupled with the proximal connector, and a distal connector positioned at the distal end of the collapsible sleeve. The instrument delivery device further includes an advancement member positioned at the distal end of the collapsible sleeve, between the distal connector and the collapsible sleeve, the advancement member comprising a housing connected to the collapsible sleeve on a first side thereof and connected to the distal connector on a second side thereof, a wheel member positioned on the housing so as to be actuatable by a user, and a roller mechanism positioned within the housing and operably connected to the wheel member such that the roller mechanism is caused to rotate responsive to actuation of the wheel member, wherein the catheter tube is routed through the roller mechanism and is engaged therewith. Rotation of the wheel member in a first direction advances the catheter tube from a first position, in which a distal end of the catheter tube is disposed within the housing or within the distal connector, to a second position, in which the distal end of the catheter tube is disposed at or adjacent a distal end of the IV catheter.

In some embodiments, the instrument delivery device further comprises a fluid delivery device coupleable to the proximal connector, the fluid delivery device configured to inject a thrombolytic agent through the proximal connector and into the lumen of the catheter tube.

In some embodiments, the proximal connector comprises a female hub coupleable to the fluid delivery device.

In some embodiments, the slit formed in the blunt tip of the catheter tube is in a normally closed position, and wherein the slit is configured to transition to an open position responsive to a fluid pressure applied to the blunt tip by the thrombolytic agent injected into the lumen of the catheter tube, with the thrombolytic agent exiting through the slit when in the open position.

In some embodiments, the distal end of the catheter tube is formed of a compliant material, and wherein a diameter of the distal end of the catheter tube expands responsive to the fluid pressure applied to the distal end by the thrombolytic agent injected into the lumen of the catheter tube.

In some embodiments, the catheter tube comprises a barrier member positioned adjacent the distal end thereof, the barrier member selectively engageable with an inner surface of the IV catheter to form a barrier that prevents a proximal flow of the thrombolytic agent back through the IV catheter.

In some embodiments, the housing comprises a vent opening formed therein, the vent opening providing for drainage of a fluid out from an interior volume of the housing.

In some embodiments, advancement of the catheter tube from the first position to the second position displaces fluid proximally out from the IV catheter, with the fluid draining out through the vent opening.

In some embodiments, the catheter tube includes positional markings thereon indicative of a positioning of the catheter tube between the first position and the second position.

In some embodiments, the collapsible sleeve is formed of a transparent material, so as to enable visualization of the positional markings on the catheter tube.

In some embodiments, the proximal connector is bonded to the proximal end of the collapsible sleeve and the proximal end of the catheter tube, such that advancement of the catheter tube toward the second position causes the proximal connector to move distally and causes the collapsible sleeve to collapse to have a shortened length.

In some embodiments, rotation of the wheel member in the first direction causes the catheter tube to advance from the first position to the second position and rotation of the wheel member in a second direction causes the catheter tube to retract toward the proximal end of the outer housing, thereby moving the instrument from the second position to the first position.

In some embodiments, the catheter tube is made of a kink-proof, fiber-optic material.

In some embodiments, the distal connector includes a blunted cannula engageable with a needle-free connector of the vascular access device and a pair of locking arms positioned on opposing side of the blunted cannula and configured to engage the needle-free connector.

Also provided herein is a method for targeted delivery of a thrombolytic agent to a distal end of an indwelling IV catheter using the instrument delivery device. The method includes coupling the distal connector to a port of a vascular access device, the port in fluid communication with the indwelling IV catheter. The method also includes coupling a fluid delivery device to the proximal connector, the fluid delivery device containing the thrombolytic agent therein. The method further includes actuating the wheel member in the first direction to advance the catheter tube from the first position to the second position to dispose the distal end of the catheter tube at or adjacent the distal end of the IV catheter and operating the fluid delivery device to inject the thrombolytic agent into the lumen of the catheter tube, with the thrombolytic agent flowing through the lumen and out through the slit formed in the blunt tip of the catheter tube, such that the thrombolytic agent is dispensed at the distal end of the indwelling IV catheter and onto a thrombotic occlusion thereat.

In some embodiments, advancement of the catheter tube from the first position to the second position proximally displaces fluid out from the IV catheter, and wherein the displaced fluid is directed proximally bask through the distal connector and into the housing, with the fluid draining out through a vent opening formed in the housing.

In some embodiments, injecting of the thrombolytic agent into the lumen of the catheter tube generates a fluid pressure within the lumen sufficient to deform the slit from a normally closed position to an open position, such that the thrombolytic agent flows out through the slit.

In some embodiments, the method further includes blocking a proximal flow of the thrombolytic agent back through the IV catheter, with proximal flow of the thrombolytic agent blocked via one of a radial expansion of the catheter tube at the distal end thereof or actuation of a barrier member at the distal end of the catheter tube.

In some embodiments, the thrombolytic agent flows out through the slit formed in the blunt tip of the catheter tube responsive to a positive pressure delivery of the thrombolytic agent by the fluid delivery device and, after delivery of the thrombolytic agent and a dissolving of the thrombotic occlusion caused thereby, the method further includes performing a negative pressure aspiration of one or more of saline, blood, a lysed occlusion, and the thrombolytic agent, back proximally through a second lumen defined between an inner wall of the IV catheter and an outer wall of the catheter tube.

Also provided herein is an instrument delivery device for targeted delivery of a thrombolytic agent to into an intravenous (IV) catheter of a vascular access device. The instrument delivery device includes a hollow spool having a distal end and a proximal end and a catheter tube wound about the hollow spool, the catheter tube defining a lumen therein and including a distal end and a proximal end, with the distal end comprising a blunt tip having a slit formed therein and the proximal end connected to the hollow spool. The instrument delivery device also includes a proximal connector connected to the proximal end of the hollow spool, a distal connector positioned at the distal end of the hollow spool, and an advancement member positioned at the distal end of the hollow spool, between the distal connector and the hollow spool. The advancement member further includes a housing connected to the hollow spool on a first side thereof and connected to the distal connector on a second side thereof, a wheel member positioned on the housing so as to be actuatable by a user, and a roller mechanism positioned within the housing and operably connected to the wheel member such that the roller mechanism is caused to rotate responsive to actuation of the wheel member, wherein the catheter tube is routed through the roller mechanism and is engaged therewith. Rotation of the wheel member in a first direction advances the catheter tube from a first position, in which a distal end of the catheter tube is disposed within the housing or within the distal connector, to a second position, in which the distal end of the catheter tube is disposed at or adjacent a distal end of the IV catheter.

In some embodiments, advancement of the catheter tube toward the second position causes the catheter tube to unwind from the hollow spool.

### BRIEF DESCRIPTION OF THE DRAWINGS

FIG. 1 is a perspective view of a system including a vascular access device, instrument delivery device and fluid delivery device, according to a non-limiting embodiment described herein;

FIG. 2 shows a side view of the instrument delivery device of FIG. 1;

FIG. 3A is a cross-sectional view of the instrument delivery device of FIG. 2, with the instrument delivery device in a first configuration;

FIG. 3B is a cross-sectional view of the instrument delivery device of FIG. 2, with the instrument delivery device in a second configuration;

FIG. 4 is a detail view of area I of FIG. 1;

FIG. 5 is a detail view of area II of FIG. 2;

FIG. 6 is a detail view of area III of FIG. 1, with a catheter tube advanced within an IV catheter in a second position;

FIG. 7 is a detail view of area III of FIG. 1, with a catheter tube advanced within an IV catheter in a second position and delivering a thrombolytic agent;

FIG. 8 is a detail view of area III of FIG. 1, with a catheter tube advanced within an IV catheter in a second position and delivering a thrombolytic agent, according to another non-limiting embodiment described herein;

FIG. 9 is a detail view of area III of FIG. 1, with a catheter tube advanced within an IV catheter in a second position and delivering a thrombolytic agent, according to another non-limiting embodiment described herein;

FIG. 10 is a detail view of area III of FIG. 1, with a catheter tube advanced within an IV catheter in a second position and delivering a thrombolytic agent, according to another non-limiting embodiment described herein;

FIG. 11A is a perspective view of a system including a vascular access device, instrument delivery device, fluid delivery device, and fluid collection device, according to another non-limiting embodiment described herein;

FIG. 11B is a perspective view of a system including a vascular access device, instrument delivery device and dual procedure syringe for fluid delivery and fluid collection, according to another non-limiting embodiment described herein;

FIG. 12 is a perspective view of an instrument delivery device, with attached fluid delivery device, according to another non-limiting embodiment described herein; and

FIG. 13 is a perspective view of an instrument delivery device, with attached fluid delivery device, according to another non-limiting embodiment described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device being manipulated by the user would be the proximal end of the device.

The terms “first”, “second”, and the like are not intended to refer to any particular order or chronology, but refer to different conditions, properties, or elements.

As used herein, “at least one of” is synonymous with “one or more of.” For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

Provided herein are devices and methods for delivering an instrument through an indwelling IV catheter, such as a peripherally inserted central catheter (PICC) or central venous catheter (CVC) as non-limiting examples, for targeted delivery of a thrombolytic agent to the tip of the IV catheter, so as to enable removal of a thrombotic occlusion. While certain vascular access devices (VADs) or catheter assemblies are discussed below and exemplified in the attached drawings with which such devices and methods may be implemented, those of skill will appreciate that any number of different VADs and/or catheter assemblies may be used within the scope of the present disclosure.

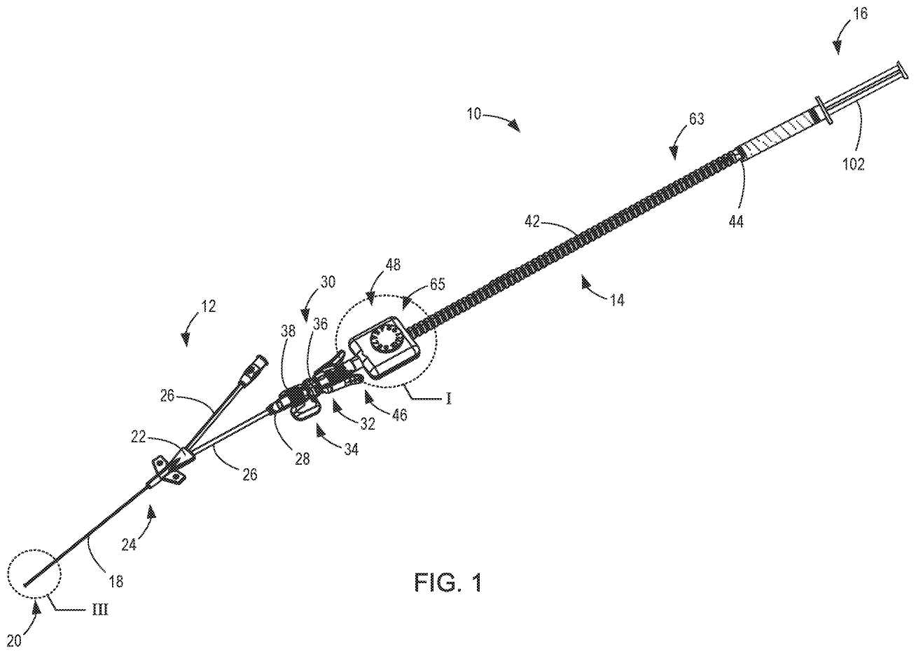

Referring now to FIG. 1, shown is a non-limiting embodiment of a system 10 including a vascular access device or catheter assembly 12, an instrument delivery device 14, and a fluid delivery device 16. The vascular access device 12 may include a catheter 18 (e.g., PICC or CVC, as non-limiting examples) having a distal end 20 that may be inserted transcutaneously through the skin of a patient at an insertion site and remain positioned therein as an indwelling IV catheter-hereafter “IV catheter 18”. The vascular access device 12 may also include a bifurcation hub 22 coupled to a proximal end 24 of the IV catheter 18 and a plurality of extension legs 26 that operably connect, via the bifurcation hub 22, to a corresponding number of lumens defined by the IV catheter 18. Each of the extension legs 26 may include a luer hub 28 positioned at a proximal end thereof. As used herein, a “luer” hub refers to a connector that includes a tapered portion (i.e., a luer taper) for creating a friction engagement between a tapered stem or elongated member of a male luer connector and a tapered cavity.

In accordance with one embodiment, the vascular access device 12 includes a catheter stabilizing catheter connector 30 (hereafter “catheter connector 30”) connected to one of luer hubs 22. In the illustrated non-limiting embodiment, the catheter connector 30 has a connector portion 32 and a stabilization portion 34. The stabilization portion 34 is configured to be placed in contact with the skin surface of a patient, to provide stability to the catheter connector 30. The connector portion 32 has a proximal port with a proximal coupler 36, a distal port with a distal coupler 38, and defines at least one lumen (not shown) extending through or otherwise in fluid communication with the couplers 36, 38. The proximal coupler 36 and/or the distal coupler 38 can be, for example, male or female luer locks and/or any other suitable coupler. In the illustrated embodiment, the proximal coupler 36 is provided as a needleless access connector (i.e., needle-free connector (NFC)) that can be physically and fluidically coupled to peripheral devices such as, instrument delivery device 14. The proximal coupler 36 presents a female luer connection/lock to which instrument delivery device 14 may be connected. The distal coupler 38 can be physically and fluidically coupled to luer hub 28, such that the lumen of the connector portion 32 is at least selectively in fluid communication with the IV catheter 18. In some embodiments, an inner surface of the connector portion 32 can be configured to provide alignment, guidance, centering, etc., to an instrument being advanced therethrough, such as by instrument delivery device 14.

The instrument delivery device 14 may be connected to the vascular access device 12 to introduce and advance an instrument into the IV catheter 18. According to aspects of the disclosure, and as explained in more detail here below, the instrument delivery device 14 may be employed for purposes of removing a thrombotic occlusion (i.e., fibrin clot) from the distal end of the IV catheter 18. That is, the instrument delivery device 14 may introduce a catheter tube into the vascular access device 12 and advance the catheter tube to an area at/adjacent the distal end of the IV catheter 18 to provide a targeted delivery of a thrombolytic agent, i.e., a tissue plasminogen activator (TPA) such as alteplase, as a non-limiting example. Upon advancement of the catheter tube to the target location, the fluid delivery device 16 may be used in connection with the instrument delivery device 14 to deliver the thrombolytic agent to the catheter tip. Delivery of the thrombolytic agent at the site of clot formation at the catheter tip results in a faster thrombolytic interaction between the agent and the fibrin clot, so to provide an effective removal of the clot.

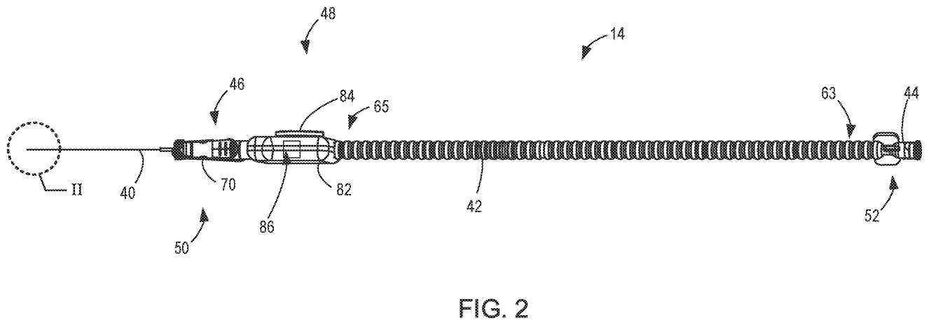

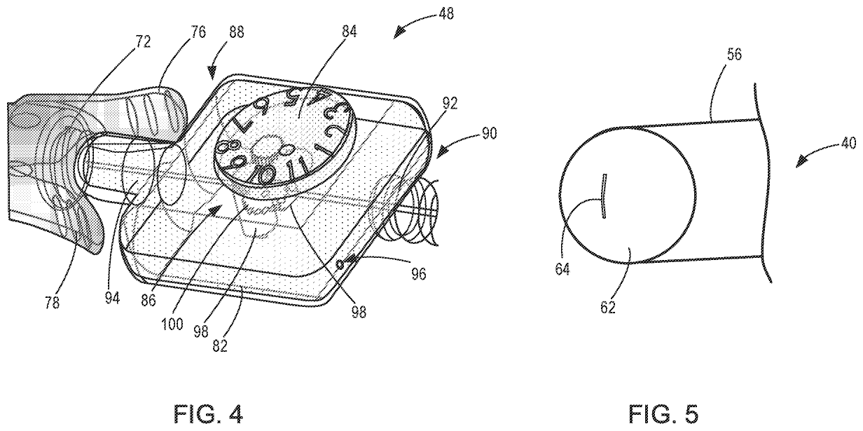

As shown in FIG. 1 and now also in FIGS. 2-7, instrument delivery device 14 includes an instrument (i.e., catheter tube) 40, a collapsible sleeve 42, a proximal connector 44, a distal connector 46, and an advancement member 48. As arranged from a distal end 50 of the instrument delivery device 14 to a proximal end 52 of the instrument delivery device 14, the distal connector 46 is provided at the distal end 50 to enable connection of the instrument delivery device 14 to the vascular access device 12 (i.e., catheter connector 30), with the advancement member 48 positioned proximal from the distal connector 46, the collapsible sleeve 42 positioned proximal from the advancement member 48, and the proximal connector 44 positioned proximal from the collapsible sleeve 42, and with the catheter tube 40 extending from the proximal connector 44, through the collapsible sleeve 42, and into/through the advancement member 48.

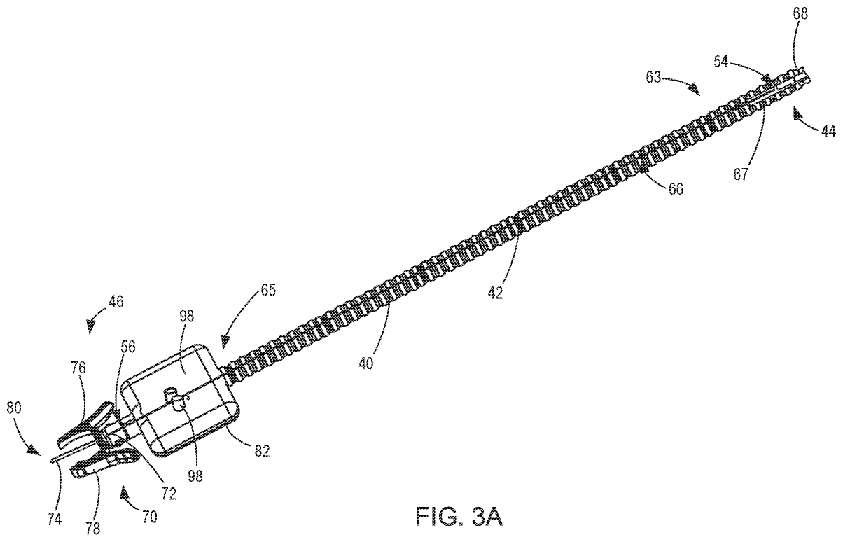

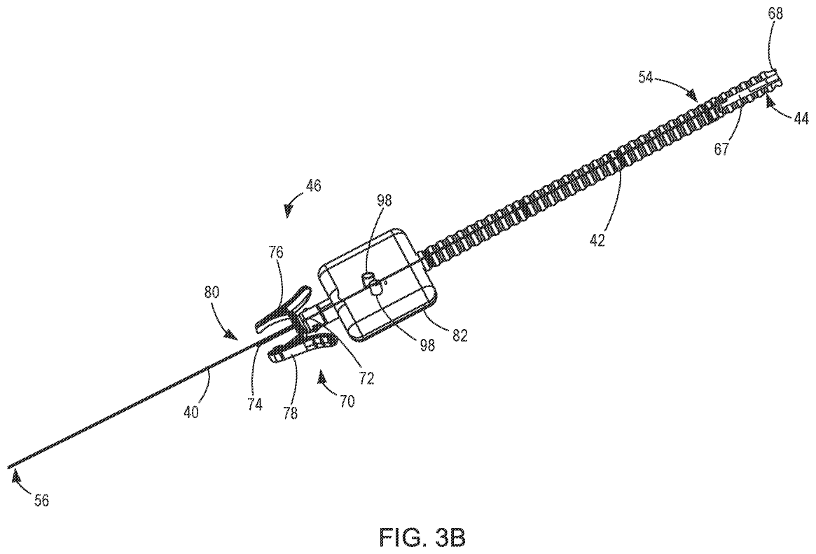

As described in further detail herein, a user can transition the instrument delivery device 14 from a first configuration to a second configuration to advance the catheter tube 40 into and through the IV catheter 18. That is, the instrument delivery device 14 can be manipulated by a user to move the catheter tube 40 from a first position as shown in FIG. 3A, in which the catheter tube 40 is entirely disposed within the instrument delivery device 14, to a second position as shown in FIG. 3B, in which at least a portion of the catheter tube 40 extends outside of the instrument delivery device 14 and into the IV catheter 18, to a location where a distal end of the catheter tube 40 is positioned at or adjacent a distal end 20 of the IV catheter 18.

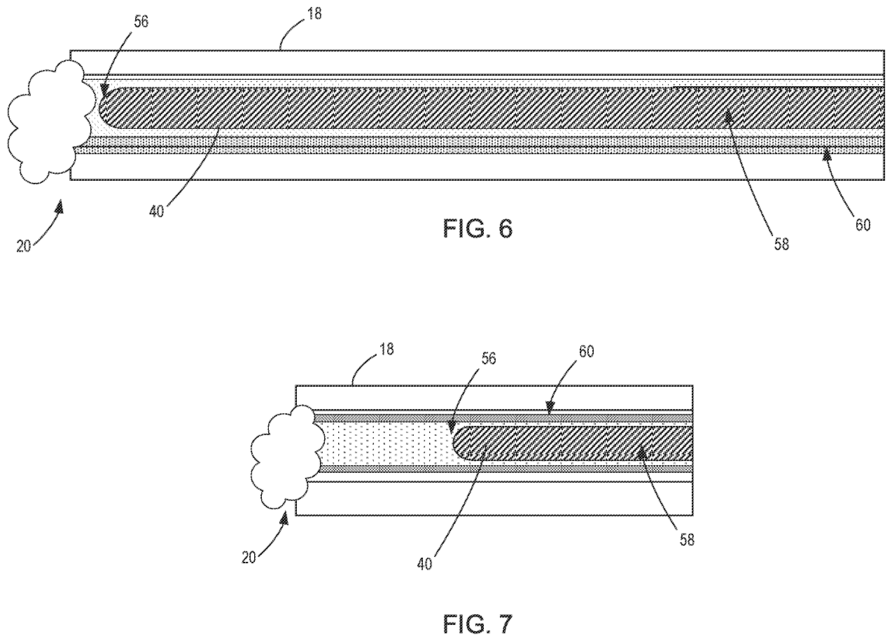

In the instrument delivery device 14, the catheter tube 40 has a proximal end 54 and a distal end 56 and defines a lumen 58 therein (see FIGS. 6 and 7). The catheter tube 40 is formed to have a smaller diameter than the IV catheter 18, such that the distal end 56 of the catheter tube 40 may be disposed within a lumen 60 (see FIGS. 6 and 7) defined by the IV catheter 18 when the catheter tube 40 is advanced into the IV catheter 18 by instrument delivery device 14. According to aspects of the disclosure, catheter tube 40 is formed of a material that makes the catheter tube 40 resistant to kinks or bowing as it is advanced during operation of the instrument delivery device 14. In non-limiting embodiments, catheter tube 40 may be formed of a polymer, such polyethylene, polypropylene, nylon, polyurethane, and/or another suitable polymeric material. In other embodiments, the catheter tube 40 may be formed of a fiber-optic material.

According to aspects of the disclosure, and as shown best in FIG. 5, the distal end 56 of the catheter tube 40 (i.e., a distal tip 56a thereof) is formed as a blunt tip 62, with the blunt tip 62 including a slit 64 formed that selectively provides for a transfer of fluid out from the lumen 58 of catheter tube 40. The slit 64 formed in the blunt tip 62 of the catheter tube 40 is configured so as to be in a normally closed position. Based on the material properties of the catheter tube 40 (i.e., of the blunt tip 62) and the configuration of the slit 64, the slit 64 is configured to transition to from the closed position to an open position responsive to a fluid pressure applied to the blunt tip 62 by fluid contained within the lumen 58 of catheter tube 40. As explained in further detail below, when fluid delivery device 16 is operated to inject fluid (e.g., a thrombolytic agent) into the catheter tube 40, the fluid applies a fluid pressure against the blunt tip 62 of the catheter tube 40 that causes the slit 64 to open, with the fluid exiting through the slit 64 when in the open position.

The collapsible sleeve 42 is formed coaxially around a (proximal) portion of the catheter tube 40 and includes a proximal end 63 and a distal end 65, with the collapsible sleeve 42 defining an inner volume 66 within which a portion of the catheter tube 40 is positioned. The proximal end 63 of the collapsible sleeve 42 is coupled to the proximal connector 44, such as via an adhesive or by implementing an ultrasonic welding process. The distal end 65 of the collapsible sleeve 42 is coupled to the advancement member 48 (i.e., to a port formed on a proximal side of a housing of the advancement member 48), such as via an adhesive or by implementing an ultrasonic welding process.

According to aspects of the disclosure, the collapsible sleeve 42 is made of a foldable and pliant material that allows the collapsible sleeve 42 to be collapsed in on itself. Thus, in some embodiments, the collapsible sleeve 42 may be made of an elastomeric or polymer film. The elastomeric or polymer film may allow the collapsible sleeve 42 to be collapsed in on itself and returned to its non-collapsed state without damage to the elastomeric or polymer film itself. In some embodiments, the collapsible sleeve 42 may include a coiled spring (not shown) formed therein to cause the collapsible sleeve 42 to be extended when a compressive force is not applied thereto. In some embodiments, the collapsible sleeve 42 may be semi or fully transparent so as to allow a clinician or other healthcare provider to view the catheter tube 40 and/or any measurement markings placed on the catheter tube 40 that are indicative of a positioning of the catheter tube 40 between the first position and the second position.

The proximal connector 44 is provided at the proximal end 52 of the instrument delivery device 14 and is bonded to the collapsible sleeve 42, as previously described. The proximal connector 44 includes a port 67 formed on a distal end thereof that is positionable within the collapsible sleeve 42 and that is sized to receive the proximal end 63 of the catheter tube 40 therein. The port 67 may form a seal with the proximal end 63 of the catheter tube 40, such that fluid may be transferred through the port 67 and to catheter tube 40 in a leak-free fashion. According to a non-limiting embodiment, the proximal end of the proximal connector 44 may be configured as a female hub 68 configured to which fluid delivery device 16 may be connected. In some embodiments, the female hub 68 is configured as a “luer” hub that includes a tapered cavity configured to receive a tapered male luer connector of fluid delivery device 16 for creating a friction engagement therebetween.

The distal connector 46 of the instrument delivery device 14 can be of any suitable shape, size, and/or configuration that enables connection of the instrument delivery device 14 to the vascular access device 12. In some embodiments, the distal connector 46 is provided as a lock 70 that includes a coupler 72, a blunt cannula 74, a first arm 76, and a second arm 78, as shown in FIGS. 2-4. In addition, the lock 70 defines a lumen 80 extending through the coupler 72 and the blunt cannula 74. The coupler 72 is configured to couple the lock 70 to the advancement member 48 (i.e., to a port formed on a distal side of a housing of the advancement member 48). The blunt cannula 74 extends from the coupler 72 and is disposed between the first arm 76 and the second arm 78. The blunt cannula 74 can be any suitable shape, size, and/or configuration. In some embodiments, the blunt cannula 74 is configured to engage a female connection of the catheter connector 30. Furthermore, the blunt cannula 74 can have an inner diameter (a diameter of a surface at least partially defining the lumen 80) that is similar to or slightly larger than an outer diameter of a portion of the catheter tube 40. Thus, the lumen 80 of the lock 70 can receive a portion of the catheter tube 40 when the instrument delivery device 14 is transitioned between the first configuration and the second configuration. With the blunt cannula 74 inserted into catheter connector 30, the first and second arms 76, 78 of the lock 70 are configured to grip opposing outer surfaces of the catheter connector 30, thereby securing the distal connector 46 (and the entire instrument delivery device 14) to the vascular access device 12.

According to aspects of the disclosure, the advancement member 48 is positioned between the distal connector 46 and the collapsible sleeve 42, to receive a distal portion of the catheter tube 40 and facilitate advancement thereof from the first position to the second position. As shown best in FIGS. 1-4, the advancement member 48 includes an outer housing 82, a wheel member 84 positioned on the housing 82 so as to be actuatable by a user, and a roller mechanism 86 positioned within the housing 82 and operably connected to the wheel member 84.

As previously described, the housing 82 of advancement member 48 includes a distal side 88 and a proximal side 90. In some embodiments, each of the distal side 88 and the proximal side 90 of housing 82 includes a port formed thereon. The port 92 on the proximal side 90 of housing 82 may be connected to the collapsible sleeve 42, to secure the housing 82 and collapsible sleeve 42 together, while the port 94 on the distal side 88 of housing 82 may be connected to the distal connector 46, to secure the housing 82 and distal connector 46 together. In some embodiments the housing 82 may be formed integrally with the distal connector 46, in which case port 94 would be eliminated (along with coupler 72).

In some embodiments, the housing 82 includes a vent opening 96 formed therein, such as in the proximal side 90 of the housing 82. The vent opening 96 provides for drainage of a fluid out from an interior volume of the housing 82, such as might occur as when the catheter tube 40 is advanced from the first position to the second position-as advancement of the catheter tube 40 displaces fluid proximally out from the IV catheter 18, through distal connector 46, and into the housing 82, with the fluid draining out through the vent opening 96.

In some embodiments, the wheel member 84 may be provided on a top surface of the housing 82 so as to be easily accessible by a clinician or other healthcare provider. The wheel member 84 may be configured as a disc-shaped dial set on the top surface of the housing 82 (i.e., laid flat on the top surface) and coupled thereto via an axle 97 about which the dial rotates. The wheel member 84 may be rotatable in a first (e.g., clockwise) direction to facilitate advancement of the catheter tube 40, with the number of rotations of the wheel member 84 calibrated to the distance travelled by the catheter tube 40. In some embodiments, the wheel member 84 may include numbering thereon that provides for an indication of the positioning of the catheter tube 40 at or between the first and second positions.

The wheel member 84 is operably connected to the roller mechanism 86 such that the roller mechanism 86 is caused to rotate responsive to actuation of the wheel member 84. In some embodiments, the roller mechanism 86 includes a pair of rollers 98 aligned in a side-by-side arrangement, with gearing 100 operably connecting the rollers 98 to the wheel member 84 and transferring rotation of the wheel member 84 to the rollers 98. The rollers 98 are positioned within housing 82 such that catheter tube 40 passes therebetween along a linear path of the catheter tube 40 between the proximal and distal connectors 44, 46. The catheter tube 40 may be gripped and engaged by the rollers 98, such that rotation of the rollers 98 (via wheel member 84) causes a linear movement of the catheter tube 40. That is, rotation of the wheel member 84 in a first direction distally advances the catheter tube 40 from the first position toward the second position.

In some embodiments, the wheel member 84 is also rotatable in a second direction (e.g., counterclockwise). Rotation of the wheel member 84 in the second direction proximally retracts the catheter tube 40 from the second position back toward the first position.

In operation of the instrument delivery device 14, an operator actuates (e.g., rotates) the wheel member 84, which in turn causes a linear movement of catheter tube 40, such as advancement or retraction of the catheter tube 40. By providing the wheel member 84 on the outer housing 82, the operator may use a single hand to both steady the instrument delivery device 14 (relative to the vascular access device 12) and operate the wheel member 84 to translate the catheter tube 40 between the first position and second position described above. Accordingly, the wheel member 84 allows for ergonomic, one-handed advancement options that allow the advancement hand to stay in a reference position without requiring significant motion axially following the motion of the catheter tube 40.

Referring still to FIGS. 1-7, operation of the system 10 for purposes of removing a thrombotic occlusion (i.e., fibrin clot) from the distal end 20 of the IV catheter 18 is now described in further detail. That is, the instrument delivery device 14 may introduce a catheter tube 40 into the vascular access device 12 and advance the catheter tube 40 to an area at/adjacent the distal end 20 of the IV catheter 18 to provide a targeted delivery of a thrombolytic agent, e.g., Alteplase, as a non-limiting example. Upon advancement of the catheter tube 40 to the target location, the fluid delivery device 16 may be used in connection with the instrument delivery device 14 to deliver the thrombolytic agent to the catheter tip, with delivery of the agent at the site of clot formation resulting in a faster thrombolytic interaction between the agent and the fibrin clot, so to provide an effective removal of the clot.

In an initial step, the instrument delivery device 14 is connected to a vascular access device 12 that has previously been placed within the vasculature of a patient. In some embodiments, a distal connector 46 of the instrument delivery device 14 that includes a blunted cannula and locking arms is connected to a catheter connector 30 (e.g., needle-free connector) of the vascular access device 12 to mechanically and fluidly connect the instrument delivery device 14 to the vascular access device 12.

Upon connection of the instrument delivery device 14 to the vascular access device 12, the wheel member 84 of the advancement member 48 is rotated in a first direction. Rotation of the wheel member 84 causes the rollers 98 of the advancement member 48 to rotate in a corresponding fashion, with the rotation of the rollers 98 pushing the catheter tube 40 (which is routed between the rollers 98) distally out from the instrument delivery device 14 and into the IV catheter 18. As the catheter tube 40 is advanced distally, the proximal connector 44 (which is connected to the proximal end 54 of catheter tube 40) is also caused to move distally. Movement of the proximal connector 44 in the distal direction causes the collapsible sleeve 42 to compress (between the proximal connector 44 and the housing 82), thereby shortening the length of the collapsible sleeve 42 and reducing the overall size/length of the instrument delivery device 14.

The method continues with additional rotation of the wheel member 84 and a corresponding distal advancement of the catheter tube 40 from the first position toward the second position. Upon a sufficient rotation of the wheel member 84 and advancement of the catheter tube 40, the distal end 56 of the catheter tube 40 reaches the site of the thrombotic occlusion at the distal end 20 of the IV catheter 18. As the catheter tube 40 is advanced to this location, the blunt tip 62 of the catheter tube 40 displaces any fluid (e.g., saline and/or heparin) contained within the lumen 60 of the IV catheter 18. In some embodiments, the displaced saline/heparin is displaced proximally back through the IV catheter 18 (and a remainder of the vascular access device 12) and to the housing 82-where the displaced fluid may be drained out from the housing 82 through vent opening 96 (and collected by an absorbent material). In some embodiments, the catheter tube 40 may then be retracted slightly (via operation of advancement member 48), to space the distal end 56 of the catheter tube 40 further proximally form the thrombotic occlusion (but still in close proximity thereto).

With the catheter tube 40 advanced to the desired second position, the fluid delivery device 16 is then connected to the proximal connector 44 of instrument delivery device 14. In some embodiments, the fluid delivery device 16 is a pre-filled syringe including a thrombolytic agent such as Alteplase contained therein. In some embodiments, the syringe 16 may include a male luer connection at the distal end thereof that connects with the female luer hub 68 of the proximal connector 44, to fluidly connect the syringe to the catheter tube 40.

Upon connection of the fluid delivery device 16 (i.e., syringe), a plunger 102 of the syringe 16 may be pressed to displace the thrombolytic agent out from the syringe under pressure, with the thrombolytic agent flowing into and through the lumen 58 of the catheter tube 40. The thrombolytic agent flows through the catheter tube 40 and reaches the distal end 56 thereof, where the pressure of the fluid causes the slit 64 in the blunted tip of the catheter tube 40 to transition from a closed to an open position. With the slit 64 open, the thrombolytic agent is able to flow out from catheter tube 40 and into the IV catheter 18 right at the location of the thrombotic occlusion-with the thrombolytic agent thus coming into direct and prolonged contact with the thrombotic occlusion. The thrombolytic agent may remain in contact with the thrombotic occlusion until the occlusion has lysed or otherwise dissolved/broken-up.

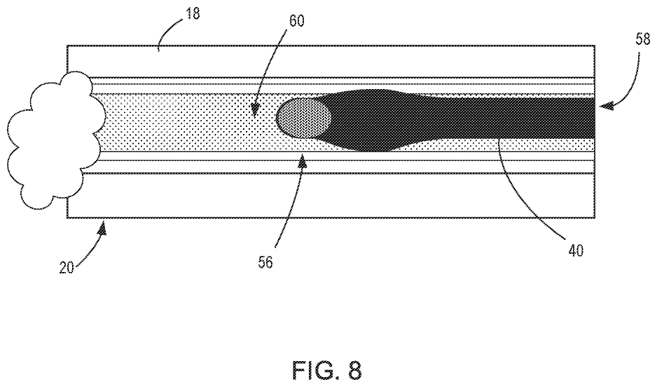

Referring now to FIGS. 8-10, additional embodiments of a catheter tube 40 that may be used in instrument delivery device 14 are shown, according to aspects of the disclosure. That is, modifications may be made to the structure of the catheter tube 40 to provide additional functionality thereto regarding the application of the thrombolytic agent to the thrombotic clot.

Referring to FIG. 8, in some embodiments, at least a portion of the distal end 56 of the catheter tube 40 is formed of a compliant material that is deformable under certain conditions. For example, when fluid delivery device 16 is operated to inject fluid (e.g., a thrombolytic agent) into the catheter tube 40, the fluid applies a fluid pressure against an inner wall of the catheter tube 40 that causes the distal end 56 of the catheter tube 40 to deform with a diameter of the distal end 56 of the catheter tube 40 expanding responsive to the fluid pressure. The increase in the diameter of the distal end 56 of the catheter tube 40 may be large enough to cause the catheter tube 40 to come into contact with the inner wall of the IV catheter 18, thereby creating a blockage that prevents the thrombolytic agent from flowing back proximally through the IV catheter 18.

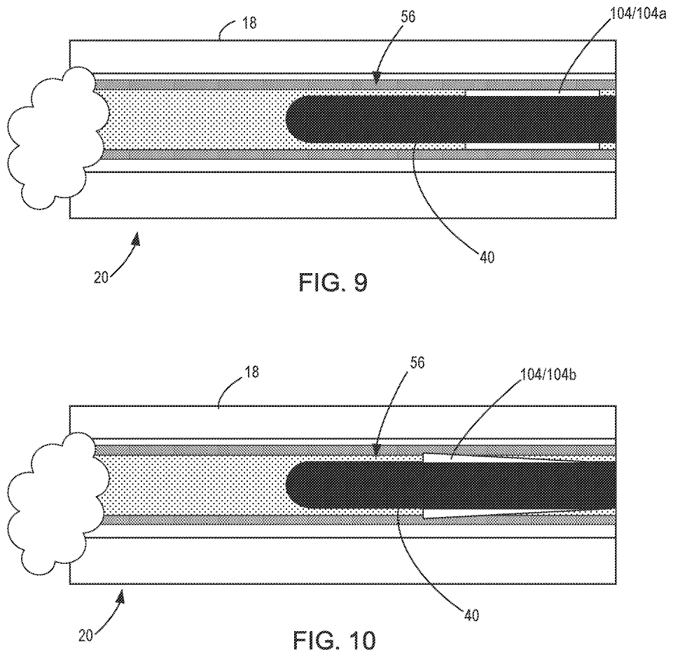

Referring to FIGS. 9 and 10, in other embodiments, the distal end 56 of the catheter tube 40 may include a separate/additional barrier member 104 positioned thereon that prevents a backward flow of the thrombolytic agent. According to embodiments, the barrier member 104 may be provided as an inflatable balloon member 104a (FIG. 9) or an umbrella-type member 104b (FIG. 10) that surrounds a location on the catheter tube 40 near the distal end 56 (e.g., 1-2 cm proximal to distal catheter tip). The barrier member 104 may be expanded outwardly so that an outer diameter thereof increases enough to come into contact with the inner wall of the IV catheter 18, thereby preventing a backward flow of the thrombolytic agent and allowing the agent to remain at the target site for a longer duration, rather than equilibrating itself over the entire IV catheter length.

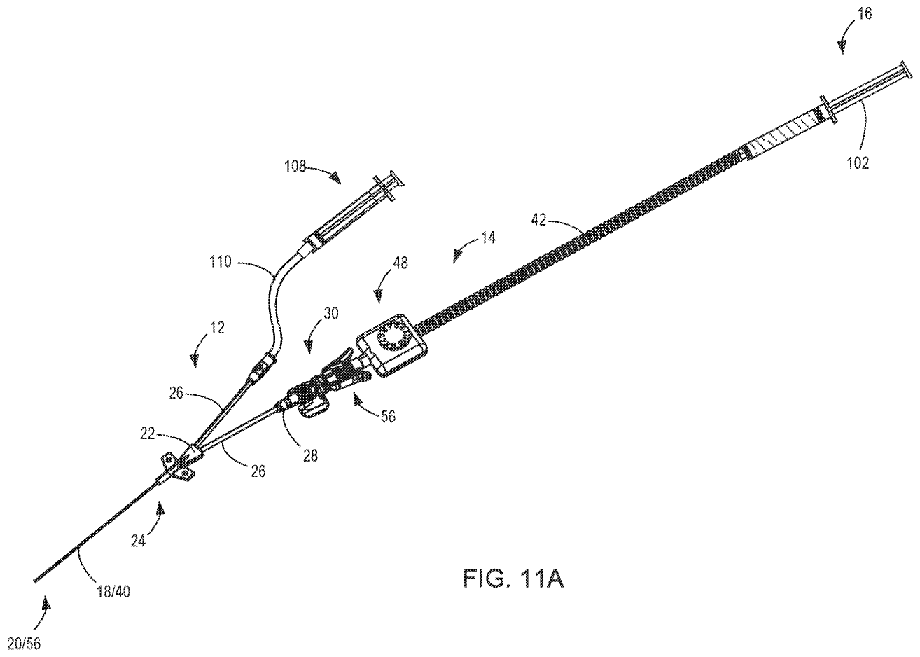

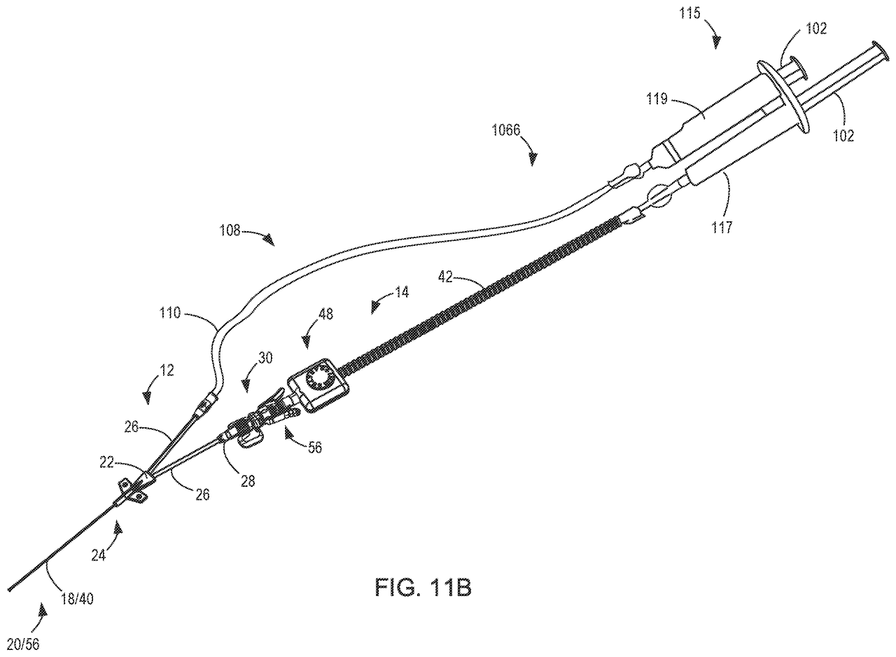

Referring now to FIGS. 11A and 11B, other embodiments of systems 106a, 106b are provided with which the instrument delivery device 14 may be employed, according to an aspect of the disclosure. In systems 106a, 106b, the structure of instrument delivery device 14 is identical to that of instrument delivery device 14 shown in FIGS. 1-7. However, as provided in detail here below, modifications may be made to the systems 106a, 106b to enable both delivery of the thrombolytic agent and aspiration of fluids (e.g., saline, blood, a lysed occlusion, and/or the thrombolytic agent) back through the IV catheter 18.

As previously described, the vascular access device 12 may include a bifurcation hub 22 coupled to a proximal end 18 of the indwelling catheter 18 and a plurality of extension legs 26 that operably connect, via the bifurcation hub 22, to a corresponding number of lumens defined by the catheter 18. Each of the extension legs 26 may include a luer hub 28 positioned at a proximal end 24 thereof.

In the embodiment of FIG. 11A, the system 106a includes each of a fluid delivery device 16 and a fluid collection device 108, with the devices connected to respective luer hubs of the vascular access device 12. The fluid delivery device 16 may be provided as a pre-filled syringe (hereafter “pre-filled syringe 16”) that acts to deliver the thrombolytic agent, while the fluid collection device 108 may be provided as an evacuated syringe or other vacutainer tube (hereafter “evacuated syringe 108”) that acts to aspirate/collect fluids. The pre-filled syringe 16 is fluidly connected to a first luer hub 28 on one of the extension legs 26 of the vascular access device 12 (through the instrument delivery device and catheter connection). The evacuated syringe 108 is fluidly connected to a second luer hub 28 on the other extension leg 20, such as via tubing 110.

In operation of the system 106a, the pre-filled syringe 16 is actuated by pressing a plunger 102 thereof to displace the thrombolytic agent out from the syringe 16 under a positive pressure, with the thrombolytic agent flowing into and through the catheter tube 40. The thrombolytic agent flows through the catheter tube 40 and reaches the distal end 56 thereof, where the pressure of the fluid causes the slit 64 in the blunted tip 62 (FIG. 5) of the catheter tube 40 to transition from a closed to an open position. With the slit 64 in the open position, the thrombolytic agent is able to flow out from catheter tube 40 and into the IV catheter 18 right at the location of the thrombotic occlusion-with the thrombolytic agent thus coming into direct and prolonged contact with the thrombotic occlusion. The thrombolytic agent may remain in contact with the thrombotic occlusion until the occlusion has lysed or otherwise dissolved/broken-up.

Upon the occlusion beginning to lyse or otherwise dissolve/break-up, the evacuated syringe 108 is actuated by proximally retracting a plunger 102 thereof, with the retracting of the plunger 102 generating a negative pressure that aspirates or draws fluid into the evacuated syringe from the IV catheter 18. That is, one or more of saline, blood, the lysed occlusion, and/or the thrombolytic agent may be aspirated into the evacuated syringe 108 responsive to generation of the negative pressure, with the fluids flowing back proximally through a “second lumen” defined between an inner wall of the IV catheter 18 and an outer wall of the catheter tube 40. The combination of the IV catheter 18 and the catheter tube 40 thus functions as a “dual mode lumen-in-lumen catheter” that provides for both fluid delivery and fluid aspiration.

In the embodiment of FIG. 11B, the system 106b includes a dual procedure syringe 115 that enables both fluid delivery and fluid collection. The dual procedure syringe 115 includes each of a delivery syringe 117 and a collection syringe 119 that are connected to respective luer hubs of the vascular access device 12. The delivery syringe 117 may be provided as a pre-filled syringe that acts to deliver the thrombolytic agent, while the collection syringe 119 may be provided as an evacuated syringe that acts to aspirate/collect fluids. The delivery syringe 117 is fluidly connected to a first luer hub 28 on one of the extension legs 26 of the vascular access device 12 (through the instrument delivery device and catheter connection). The collection syringe 119 is fluidly connected to a second luer hub 28 on the other extension leg 20, such as via tubing 110.

In operation of the dual procedure syringe 115, the delivery syringe 117 is actuated by pressing a plunger 102 thereof to displace the thrombolytic agent out from the delivery syringe 117 under a positive pressure, with the thrombolytic agent flowing into and through the catheter tube 40. The thrombolytic agent flows through the catheter tube 40 and reaches the distal end 56 thereof, where the pressure of the fluid causes the slit 64 in the blunted tip 62 (FIG. 5) of the catheter tube 40 to transition from a closed to an open position. With the slit 64 in the open position, the thrombolytic agent is able to flow out from catheter tube 40 and into the IV catheter 18 right at the location of the thrombotic occlusion-with the thrombolytic agent thus coming into direct and prolonged contact with the thrombotic occlusion.

Actuation of the delivery syringe 117 causes a corresponding actuation of the collection syringe 119, with a plunger 102 of the collection syringe 119 retracting responsive to advancement of the plunger 102 of delivery syringe 119. The retracting of the plunger 102 of the collection syringe 119 generates a negative pressure that aspirates or draws fluid into the collection syringe 119 from the IV catheter 18. That is, one or more of saline, blood, the lysed occlusion, and/or the thrombolytic agent may be aspirated into the collection syringe 119 responsive to generation of the negative pressure, with the fluids flowing back proximally through a “second lumen” defined between an inner wall of the IV catheter 18 and an outer wall of the catheter tube 40. The combination of the IV catheter 18 and the catheter tube 40 thus functions as a “dual mode lumen-in-lumen catheter” that provides for both fluid delivery and fluid aspiration.

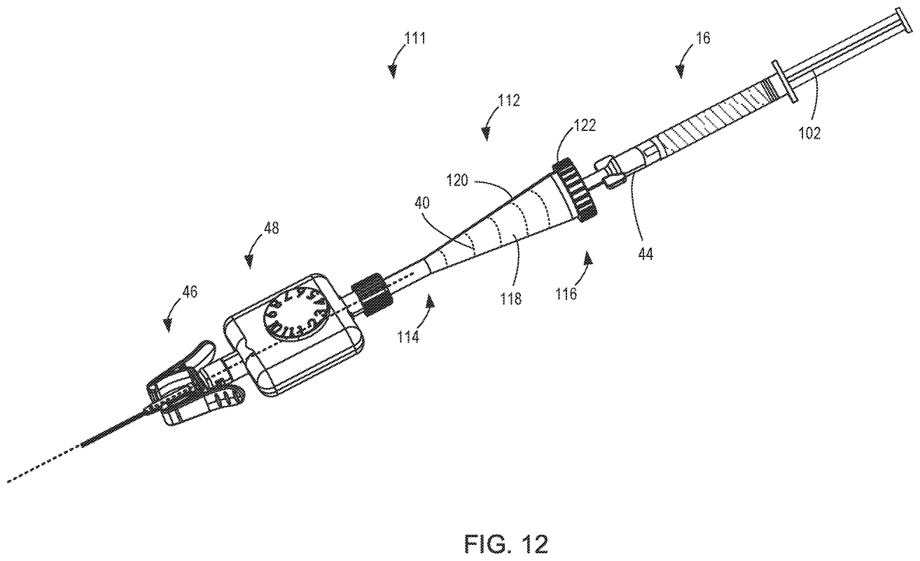

Referring now to FIG. 12, another embodiment of an instrument delivery device 111 is shown, according to an aspect of the disclosure. The structure of instrument delivery device 111 is substantially similar to that of instrument delivery device 14 shown in FIGS. 1-7, and thus like members are labeled identically in instrument delivery device 111. However, as provided in detail here below, the collapsible sleeve 42 of instrument delivery device 14 is replaced by a spool 112 in instrument delivery device 111, about which the catheter tube 40 may be wound.

The spool 112 includes a distal end 114 and a proximal end 116, and includes a hollow inner spool member 118, a cover member 120 positioned over the inner spool member 118, and an end cap 122 positioned at the proximal end 116 of the spool member 118. The spool 112 is positioned between the advancement member 48 and the proximal connector 44 such that the distal end 114 of the spool 112 is connected to the housing 82 of advancement member 48 (such as via a male luer connection of cover member 120 mating with a female luer on housing 82) and the proximal end 116 is connected to the proximal connector 44 (such as via a coupling of end cap 122 to proximal connector 44). The inner spool member 118 may have a conical-type construction whose diameter expands from the distal end to the proximal end, and the catheter tube 40 may be routed within a hollow interior of the inner spool member 118, extending through openings at each of the distal and proximal ends. The catheter tube 40 may be wound about an inner surface of the inner spool member 118 and, in one embodiment, may be retained within grooves formed in the inner surface.

In operation of the instrument delivery device 111, the wheel member 84 of the advancement member 48 is rotated in a first direction. Rotation of the wheel member 84 causes the rollers 98 of the advancement member 48 to rotate in a corresponding fashion, with the rotation of the rollers 98 pushing the catheter tube 40 (which is routed between the rollers 98) distally out from the instrument delivery device 111 and into the IV catheter 18-to transition the catheter tube 40 from the first position toward the second position. In being advanced distally, the catheter tube 40 unwinds/uncoils from the spool member 118, so as to allow for the distal advancement of the catheter tube 40.

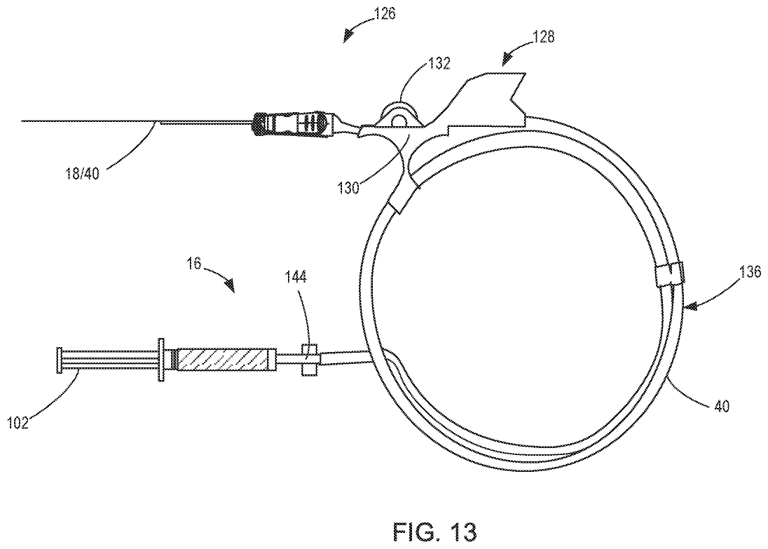

Referring now to FIG. 13, another embodiment of an instrument delivery device 126 is shown, according to an aspect of the disclosure. The structure of instrument delivery device 126 differs from those of instrument delivery devices 14, 111 regarding the replacement of advancement member 48 and collapsible sleeve 42 with other substitute members. As shown in FIG. 13, instrument delivery device 126 includes a graspable body 128 that includes a housing 130 and a wheel member 132 provided on a top surface of the housing 130, with the wheel member 132 oriented orthogonal to the top surface. The wheel member 132 may be partially housed within housing 130 (i.e., within a compartment formed in the housing 130) and configured to extend upwardly from the housing 130, so as to be engageable by a single finger (e.g., thumb) of an operator. An operator can directly rotate the wheel member 132 in a forward direction (i.e., toward the distal end) or in a backward direction (i.e., toward the proximal end) to advance or retract the catheter tube 40 between the proximal and distal connectors 44, 46—with rotation of the wheel member 132 in the forward direction distally advancing the catheter tube 40 from the first position toward the second position.

The instrument delivery device 126 also includes a coiled housing member 136 having a distal end 114 connected to the housing 130 of graspable body 128 and a proximal end connected to the proximal connector 44. The catheter tube 40 may be routed within a hollow interior of the coiled housing member 136, extending through openings at each of the distal and proximal ends.

In operation of the instrument delivery device 126, the wheel member 132 is rotated in a forward direction. Rotation of the wheel member 132 pushes the catheter tube 40 distally out from the instrument delivery device 126 and into the IV catheter 18—to transition the catheter tube 40 from the first position toward the second position. In being advanced distally, the catheter tube 40 unwinds/uncoils from the spool member 118, so as to allow for the distal advancement of the catheter tube 40.

Beneficially, embodiments of the instrument delivery devices described herein provide for introduction of a catheter tube into a vascular access device and advancement of the catheter tube to an area at/adjacent the distal tip of an indwelling IV catheter, to provide a targeted delivery of a thrombolytic agent (e.g., Alteplase) to a thrombotic occlusion that has formed thereat. Upon advancement of the catheter tube to the target location, a fluid delivery device may be connected to the instrument delivery device to deliver the thrombolytic agent to the catheter tip. Delivery of the thrombolytic agent at the site of clot formation at the catheter tip results in a faster thrombolytic interaction between the agent and the fibrin clot, so to provide an effective removal of the clot.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A system comprising:
a vascular access device comprising an intravenous (IV) catheter; and
an instrument delivery device for targeted delivery of a thrombolytic agent into the IV catheter, the instrument delivery device comprising:
a collapsible sleeve comprising a distal end and a proximal end;
a catheter tube positioned at least partially within the collapsible sleeve and coaxially therewith, the catheter tube defining a lumen therein and including a distal end and a proximal end, the distal end comprising a blunt tip having a slit formed therein;
a proximal connector positioned at the proximal end of the collapsible sleeve, with the proximal end of the collapsible sleeve and the proximal end of the catheter tube coupled with the proximal connector;
a distal connector positioned at the distal end of the collapsible sleeve; and
an advancement member positioned at the distal end of the collapsible sleeve, between the distal connector and the collapsible sleeve, the advancement member comprising:
a housing connected to the collapsible sleeve on a first side thereof and connected to the distal connector on a second side thereof;
a wheel member positioned on the housing so as to be actuatable by a user; and
a roller mechanism positioned within the housing and operably connected to the wheel member such that the roller mechanism is caused to rotate responsive to actuation of the wheel member, wherein the catheter tube is routed through the roller mechanism and is engaged therewith;
wherein rotation of the wheel member in a first direction advances the catheter tube from a first position, in which a distal end of the catheter tube is disposed within the housing or within the distal connector, to a second position, in which the distal end of the catheter tube is disposed at or adjacent a distal end of the IV catheter.

2. The system of claim 1, further comprising a fluid delivery device coupleable to the proximal connector, the fluid delivery device configured to inject a thrombolytic agent through the proximal connector and into the lumen of the catheter tube.

3. The system of claim 2, wherein the proximal connector comprises a female hub coupleable to the fluid delivery device.

4. The system of claim 2, wherein the slit formed in the blunt tip of the catheter tube is in a normally closed position, and wherein the slit is configured to transition to an open position responsive to a fluid pressure applied to the blunt tip by the thrombolytic agent injected into the lumen of the catheter tube, with the thrombolytic agent exiting through the slit when in the open position.

5. The system of claim 4, wherein the distal end of the catheter tube is formed of a compliant material, and wherein a diameter of the distal end of the catheter tube expands responsive to the fluid pressure applied to the distal end by the thrombolytic agent injected into the lumen of the catheter tube.

6. The system of claim 4, wherein the catheter tube comprises a barrier member positioned adjacent the distal end thereof, the barrier member selectively engageable with an inner surface of the IV catheter to form a barrier that prevents a proximal flow of the thrombolytic agent back through the IV catheter.

7. The system of claim 1, wherein the housing comprises a vent opening formed therein, the vent opening providing for drainage of a fluid out from an interior volume of the housing.

8. The system of any of claim 7, wherein advancement of the catheter tube from the first position to the second position displaces fluid proximally out from the IV catheter, with the fluid draining out through the vent opening.

9. The system of claim 1, wherein the catheter tube includes positional markings thereon indicative of a positioning of the catheter tube between the first position and the second position.

10. The system of claim 9, wherein the collapsible sleeve is formed of a transparent material, so as to enable visualization of the positional markings on the catheter tube.

11. The system of claim 1, wherein the proximal connector is bonded to the proximal end of the collapsible sleeve and the proximal end of the catheter tube, such that advancement of the catheter tube toward the second position causes the proximal connector to move distally and causes the collapsible sleeve to collapse to have a shortened length.

12. The system of claim 1, wherein rotation of the wheel member in the first direction causes the catheter tube to advance from the first position to the second position; and
wherein rotation of the wheel member in a second direction causes the catheter tube to retract toward the proximal end of the outer housing, thereby moving the instrument from the second position to the first position.

13. The system of claim 1, wherein the catheter tube is made of a kink-proof, fiber-optic material.

14. The system of claim 1, wherein the distal connector comprises:
a blunted cannula engageable with a needle-free connector of the vascular access device; and
a pair of locking arms positioned on opposing side of the blunted cannula and configured to engage the needle-free connector.

15. A method for targeted delivery of a thrombolytic agent to a distal end of an indwelling IV catheter, the method comprising:
providing a vascular access device comprising an intravenous (IV) catheter and an instrument delivery device for targeted delivery of a thrombolytic agent into the IV catheter, the instrument delivery device comprising:
a collapsible sleeve comprising a distal end and a proximal end;
a catheter tube positioned at least partially within the collapsible sleeve and coaxially therewith, the catheter tube defining a lumen therein and including a distal end and a proximal end, the distal end comprising a blunt tip having a slit formed therein;
a proximal connector positioned at the proximal end of the collapsible sleeve, with the proximal end of the collapsible sleeve and the proximal end of the catheter tube coupled with the proximal connector;
a distal connector positioned at the distal end of the collapsible sleeve; and
an advancement member positioned at the distal end of the collapsible sleeve, between the distal connector and the collapsible sleeve, the advancement member comprising:
a housing connected to the collapsible sleeve on a first side thereof and connected to the distal connector on a second side thereof;
a wheel member positioned on the housing so as to be actuatable by a user; and
a roller mechanism positioned within the housing and operably connected to the wheel member such that the roller mechanism is caused to rotate responsive to actuation of the wheel member, wherein the catheter tube is routed through the roller mechanism and is engaged therewith;
coupling the distal connector to a port of the vascular access device, the port in fluid communication with the indwelling IV catheter;
coupling a fluid delivery device to the proximal connector, the fluid delivery device containing the thrombolytic agent therein;
actuating the wheel member in the first direction to advance the catheter tube from the first position to the second position to dispose the distal end of the catheter tube at or adjacent the distal end of the IV catheter; and
operating the fluid delivery device to inject the thrombolytic agent into the lumen of the catheter tube, with the thrombolytic agent flowing through the lumen and out through the slit formed in the blunt tip of the catheter tube, such that the thrombolytic agent is dispensed at the distal end of the indwelling IV catheter and onto a thrombotic occlusion thereat.

16. The method of claim 15, wherein advancement of the catheter tube from the first position to the second position proximally displaces fluid out from the IV catheter, and wherein the displaced fluid is directed proximally bask through the distal connector and into the housing, with the fluid draining out through a vent opening formed in the housing.

17. The method of claim 15, wherein injecting of the thrombolytic agent into the lumen of the catheter tube generates a fluid pressure within the lumen sufficient to deform the slit from a normally closed position to an open position, such that the thrombolytic agent flows out through the slit.

18. The method of claim 15, further comprising blocking a proximal flow of the thrombolytic agent back through the IV catheter, with proximal flow of the thrombolytic agent blocked via one of a radial expansion of the catheter tube at the distal end thereof or actuation of a barrier member at the distal end of the catheter tube.

19. The method of claim 15, wherein the thrombolytic agent flows out through the slit formed in the blunt tip of the catheter tube responsive to a positive pressure delivery of the thrombolytic agent by the fluid delivery device; and
wherein after delivery of the thrombolytic agent and a dissolving of the thrombotic occlusion caused thereby, the method further comprises performing a negative pressure aspiration of one or more of saline, blood, a lysed occlusion, and the thrombolytic agent, back proximally through a second lumen defined between an inner wall of the IV catheter and an outer wall of the catheter tube.

20. A system comprising:
a vascular access device comprising an intravenous (IV) catheter; and
an instrument delivery device for targeted delivery of a thrombolytic agent into the IV catheter, the instrument delivery device comprising:
a hollow spool including a distal end and a proximal end;
a catheter tube wound about the hollow spool, the catheter tube defining a lumen therein and including a distal end and a proximal end, the distal end comprising a blunt tip having a slit formed therein and the proximal end connected to the hollow spool;
a proximal connector connected to the proximal end of the hollow spool;
a distal connector positioned at the distal end of the hollow spool; and
an advancement member positioned at the distal end of the hollow spool, between the distal connector and the hollow spool, the advancement member comprising:
a housing connected to the hollow spool on a first side thereof and connected to the distal connector on a second side thereof;
a wheel member positioned on the housing so as to be actuatable by a user; and
a roller mechanism positioned within the housing and operably connected to the wheel member such that the roller mechanism is caused to rotate responsive to actuation of the wheel member, wherein the catheter tube is routed through the roller mechanism and is engaged therewith;
wherein rotation of the wheel member in a first direction advances the catheter tube from a first position, in which a distal end of the catheter tube is disposed within the housing or within the distal connector, to a second position, in which the distal end of the catheter tube is disposed at or adjacent a distal end of the IV catheter.

21. The system of claim 20, wherein advancement of the catheter tube toward the second position causes the catheter tube to unwind from the hollow spool.
